(19) United States
(12) Patent Application Publication
(10) Pub. No.: US 2017/0049307 A1
(43) Pub. Date: Feb. 23, 2017

(54) VIDEO ENDOSCOPE

(71) Applicant: OLYMPUS WINTER & IBE GMBH, Hamburg (DE)

(72) Inventor: Martin WIETERS, Barsbuettel (DE)

(73) Assignee: OLYMPUS WINTER & IBE GMBH, Hamburg (DE)

(21) Appl. No.: 15/343,271

(22) Filed: Nov. 4, 2016

Related U.S. Application Data
(63) Continuation of application No. PCT/EP2015/059571, filed on Apr. 30, 2015.

(30) Foreign Application Priority Data
May 8, 2014 (DE) 102014208652.3

(51) Int. Cl. A61B 1/05; A61B 1/005; A61B 1/00

(52) U.S. Cl. CPC A61B 1/051; A61B 1/00006; A61B 1/00096; A61B 1/0057; A61B 1/0058; A61B 1/00188

(57) ABSTRACT

A video endoscope including: an endoscope shaft; an optical assembly provided in a distal region of the endoscope shaft, the optical assembly being mounted in the endoscope shaft by a mounting; and one or more reversibly deformable bending actuators operatively connected to the mounting such that the one or more bending actuators move the optical assembly relative to the endoscope shaft.

### CROSS-REFERENCE TO RELATED APPLICATION

The present application is a continuation of PCT/EP2015/059571 filed on Apr. 30, 2015, which is based upon and claims the benefit to DE 10 2014 208 652.3 filed on May 8, 2014, the entire contents of each of which are incorporated herein by reference.

### BACKGROUND

Field

The present application relates to a video endoscope comprising an endoscope shaft, wherein an optical assembly is provided in the distal region in the endoscope shaft, wherein the assembly is mounted in the endoscope shaft by means of a mounting for the assembly.

Prior Art

In this disclosure, the term “video endoscopes” is understood as endoscopes combined with at least one image sensor which is configured for taking a video recording, irrespective of whether the image sensor is arranged distally in the endoscope shaft, proximally in a grip or externally in a camera head which is able to be positioned on an eyepiece in the proximal region of the endoscope, i.e. on the side of an operator.

Moreover, video endoscopes with a lateral viewing direction are also known, i.e. a viewing direction which is not equal to 0°, in which a distal deflection prism of a first optical assembly is received by an outer tube and the image sensor unit of the second optical group, in particular the CCD image sensor unit, is arranged in an inner tube. In this case, the two tubes for the optical assemblies are tensioned in the proximal grip region by a spring, wherein in the distal region of the endoscope shaft a radial bearing is provided between the outer tube and the inner tube.

In FIG. 1 an endoscope 1 known from the prior art is shown schematically. The endoscope 1 has at the proximal end, shown to the right, a grip 3 which leads into an endoscope shaft 2. The distal end of the endoscope shaft 2 is shown on the left-hand side in FIG. 1.

The grip 3 has a rotary ring 4, the inner tube 7 being able to be rotated thereby relative to an outer tube 6 via bar magnets 5 which are connected to the inner tube 7, in order to alter the viewing direction of the endoscope 1. The inner tube 7 is also mounted in the grip 3 by means of a radial bearing 8. Moreover, the grip 3 has a pretensioning apparatus consisting of a compression spring 9 which is pretensioned relative to a stop 10 for the compression spring 9. The compression spring 9 ensures that the inner tube 7 is pressed and/or pretensioned in the axial direction toward the distal end 11 of the endoscope shaft 2.

At the distal end 11, the endoscope shaft 2 has an aperture 12 which faces laterally. An optical assembly 13 with lenses and prisms is located behind the aperture 12, the light entering through the aperture 12 being deflected thereby in a direction parallel to the longitudinal axis of the endoscope shaft 2. The optical assembly 13 is mounted by a mounting 14 which is connected to the outer tube 6. The aperture 12 is also part of the optical assembly 13.

Proximally, a second optical assembly 16 adjoins the first optical assembly 13, said second optical assembly in this case terminating in an image sensor unit 19. The second optical assembly 16 is mounted in a mounting 17 which is connected to the inner tube 7 such that it performs rotations or displacements of the inner tube 7 therewith. The inner tube 7 is mounted radially relative to the outer tube 6 in the region of the distal end 11 of the endoscope shaft 2, by means of a radial bearing 18.

The distal front surface of the mounting 17 of the second optical assembly 16 and the proximal front surface of the mounting 14 of the first optical assembly 13 are arranged opposite one another and form an axial bearing 15. By the pretensioning of the inner tube 7 in the axial direction by the compression spring 9 in the grip 3 the axial bearing 15 is closed, i.e. the distal front surface of the mounting 17 is pressed against the proximal front surface of the mounting 14. As a result, the axial position of the second optical assembly 16 is fixedly defined relative to the first optical assembly 13.

### SUMMARY

Proceeding from the prior art, an object is to improve a mounted arrangement of an assembly in the endoscope shaft of an endoscope in a simple manner.

This object can be achieved by a video endoscope comprising an endoscope shaft, wherein an optical assembly is provided in the distal region in the endoscope shaft, wherein the assembly is mounted in the endoscope shaft by means of a mounting for the assembly, said video endoscope being developed such that the mounting for the assembly has at least one or more reversibly deformable bending actuators, wherein the bending actuator or the bending actuators is or are designed to move the assembly during a movement of the bending actuator or the bending actuators, preferably relative to a longitudinal axis or the longitudinal axis of the endoscope shaft.

By means of one or more bending actuators for the mounting of an optical assembly in the endoscope shaft it is possible to position and to align the assembly by deflecting the bending actuators. If, for example, the optical assembly is configured as an image sensor unit, it is thereby possible, for example, to adapt and to modify the spacing between an objective lens and the image sensor unit mounted by means of the bending actuators. As a result, it is achieved that the image sensor unit may be displaced and/or may be tilted relative to the objective lens in the endoscope shaft and/or relative to the longitudinal axis of the endoscope shaft, whereby it is possible to compensate for tolerances during the manufacture and mounting of the assemblies.

By means of the bending actuators provided for mounting the assembly it is also possible to utilize effectively the small constructional space between the inner wall of the endoscope shaft and the assembly.

Moreover, in one embodiment of the endoscope, at least one or more, such as two or three or four, bending actuators are arranged between the inner wall of the endoscope shaft and the assembly mounted by the bending actuators and/or in that the bending actuators are arranged in the endoscope shaft symmetrically and/or uniformly relative to a longitudinal axis of the endoscope shaft. By the use of a plurality of bending actuators, it is possible by deflecting the bending actuators relative to the longitudinal axis of the endoscope shaft and/or relative to the optical axis of an optical assembly to move the assembly such that the spacing relative to a second optical assembly and the assembly moved by the bending actuators is adjusted. Moreover, the optical axis, for example of the optical assembly moved by the bending actuators, may also be tilted and/or adjusted relative to the longitudinal axis of the endoscope shaft. Moreover, by means of the bending actuators it is ensured that the mounted assembly is and/or is being securely mounted and aligned in the endoscope shaft. For example, the bending actuators in each case can be configured to be bar-shaped or as a bar-shaped element which may be shifted and which, with a corresponding activation by a control device, follows a predetermined adjustment path or produces a predetermined adjusting force. Moreover, the bending actuators can be reversibly deformable so that, after a deflection of the bending actuators, the assembly which has been moved and deflected thereby is moved back into its initial or original position.

The bending actuators can be aligned radially inwardly relative to the longitudinal axis of the endoscope shaft, resulting in a star-shaped arrangement of the bending actuators.

In one embodiment, the bending actuator or the bending actuators in each case can be connected at the end to the optical assembly, forming an adhesively bonded contact point between the respective bending actuator and the assembly. As a result, the bending actuators can be permanently and securely connected by means of adhesive bonding to the assembly to be moved.

Moreover, according to one embodiment, the bending actuator or the bending actuators can be arranged on a partially circular or an annular mounting body. For example, the mounting body can be configured as a ring, so that on the ring the bending actuators are arranged in a star-shaped manner inwardly, i.e. toward the centre point of the ring and toward the longitudinal axis of the endoscope shaft.

The bending actuator or the bending actuators in each case can be configured as planar and/or plate-shaped and/or strip-shaped bending actuators.

Moreover, in one embodiment, the at least one bending actuator or the bending actuators can be configured as a piezo-electric bending actuator, as a bending actuator with a shape memory alloy or with a bimetal arrangement.

Moreover, a control device can be provided for the at least one bending actuator or for the bending actuators, wherein the bending actuators can be activated by means of the control device, such as being individually activated. The control device can be provided outside the endoscope shaft, wherein the control device is connected to the bending actuators in the inside of the endoscope shaft via corresponding leads.

In one embodiment, the assembly can be configured as an optical assembly with at least one lens and/or with a prism and/or with an objective lens.

Moreover, a further embodiment of the endoscope can be configured as an image sensor unit having an image sensor, such as CCD sensor or a CMOS sensor. Since the position of an image sensor unit inside the endoscope shaft is aligned in an adjustable manner by means of bending actuators, this results in a simple handling of the image sensor unit which can be configured as a rectangular component. By the mounting and simultaneous movement of the image sensor unit by means of the bending actuators it is possible, for example, to adjust in a simple manner the spacing between an objective lens and the image sensor unit (as image recorder) during use. As a result, for example, an improvement of the depth of field is also achieved if the optimal spacing between the objective lens and the image sensor unit is achieved by a movement of the image sensor unit by means of the bending actuators.

Further features will become apparent from the description of embodiments, together with the claims and the accompanying drawings. The embodiments may implement individual features or a combination of a plurality of features.

### BRIEF DESCRIPTION OF THE DRAWINGS

Exemplary embodiments are disclosed herein with reference to the drawings, without limiting the general inventive idea, wherein relative to all of the details not described in more detail in the text, reference is expressly made to the drawings, in which:

FIG. 1 illustrates a schematic view of an endoscope according to the prior art.

FIG. 2a illustrates a schematic cross section through an arrangement in cross section formed by an image sensor unit and an optical assembly.

FIG. 2b illustrates a schematic plan view of the arrangement of FIG. 2a.

FIG. 2c illustrates a perspective view of the arrangement of FIG. 2a, which is arranged in an endoscope shaft of an endoscope.

In the drawings, in each case the same or similar elements and/or parts are provided with the same reference numerals, so that in each case a corresponding re-introduction is dispensed with.

### DETAILED DESCRIPTION

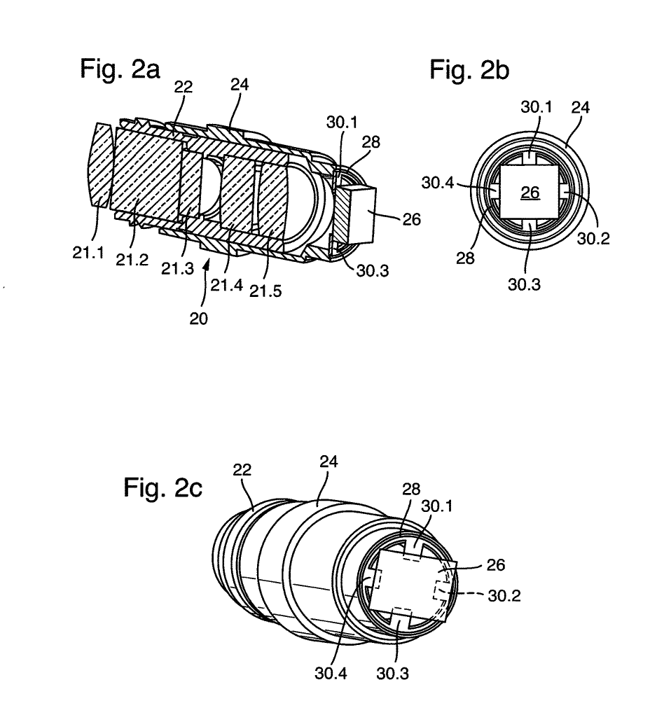

Turning first to FIG. 2a, an optical arrangement for an endoscope shaft is shown schematically in cross section, wherein the arrangement as an optical first assembly 20 has a lens system with a plurality of lenses 21.1 to 21.5. The lenses 21.1 to 21.5 of the lens system 20 are arranged in a sleeve-like mounting or sleeve 22. The arrangement shown is arranged, for example, in the distal end of an endoscope shaft (not shown here).

The sleeve 22 with the lens arrangement and/or the lens system is surrounded by a further sleeve 24, an image sensor 26 being arranged at the end thereof remote from the lens system in order to record the light beams passed through the lenses 21.1 to 21.5 of the lens system of the first optical assembly.

The image sensor unit 26 is mounted by means of bending actuators 30.1, 30.2, 30.3, 30.4 arranged on a ring 28. The ring 28 in this case is arranged so as to bear against the inner face of the sleeve 24 in a receiver and surrounds the image sensor unit 26.

In FIG. 2b a plan view of the image sensor unit 26 and the bending actuators 30.1 to 30.4 connected to the image sensor unit 26 is shown. In the perspective view shown in FIG. 2c, for reasons of improved clarity, the image sensor 26 is shown as transparent.

The bending actuators 30.1 to 30.4 are arranged in a star-shaped manner and protruding uniformly inwardly, i.e. protruding inwardly toward the central longitudinal axis of the endoscope shaft (not shown here), wherein at the ends of the plate-shaped elongated bending actuators 30.1 to 30.4, the image sensor 26 is connected in the edge region to the bending actuators 30.1 to 30.4. For example, the bending actuators 30.1 to 30.4 are of cuboidal shape, and consist of a plurality of piezo-electrical layers so that by using the piezo-electrical effect and a corresponding activation of the bending actuators by means of a control device (not shown here), the bending actuators 30.1 to 30.4 are deflected in a controlled manner, whereby the optical axis of the image sensor unit 26 is oriented in a tilted manner, for example, relative to the longitudinal axis of an endoscope shaft.

The actuators may, therefore, be provided with sensors, not shown, the controller and/or the control device being able to determine thereby the current deflection of the actuators.

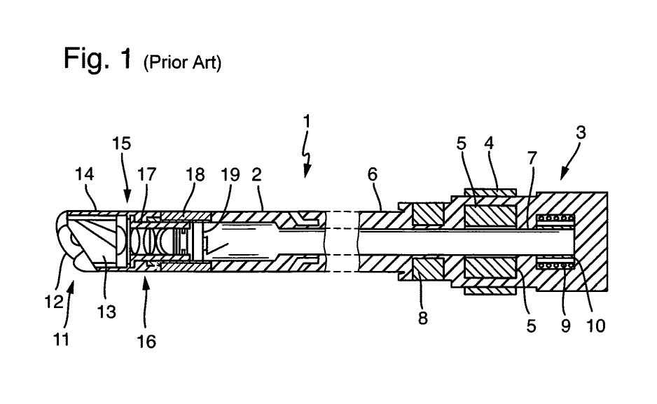

The arrangement shown in FIGS. 2a to 2c with the lens system and the image sensor unit 26 in this case can be arranged in the distal region of an endoscope shaft (similarly to that the optical assembly 16 shown in FIG. 1).

In one embodiment, the bending actuators 30.1 to 30.4, for example, are produced as a shape memory alloy or consist of bimetals so that the bending actuators 30.1 to 30.4 are also reversibly movable, in order to move the location or respectively position of the image sensor unit 26 relative to the first optical assembly configured with the lens system. By a deflected movement of the bending actuators 30.1 to 30.4 in a controlled manner the spacing between the lens system and the image sensor unit is altered and adapted.

By the movably mounted arrangement of the image sensor unit 26 in the endoscope shaft which is configured, for example, as a CCD sensor, error tolerances during the manufacture of an endoscope can be compensated in a simple manner by adjusting the spacing between the lens system and the image sensor unit 26.

While there has been shown and described what is considered to be preferred embodiments, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### List of Reference Numerals

1 Endoscope
2 Endoscope shaft
3 Grip
4 Rotary ring
5 Bar magnet
6 Outer tube
7 Inner tube
7a Inner tube portion
8 Radial bearing
9 Compression spring
10 Stop for compression spring
11 Distal end
12 Aperture
13 Optical assembly comprising lenses and prisms
14 Mounting of optical assembly
15 Axial bearing
16 Optical assembly
17 Mounting of optical assembly
18 Radial bearing
19 Image sensor unit
20 Arrangement
21.1 to 21.5 Lens
22 Sleeve
24 Sleeve
26 Image sensor unit
28 Ring
30.1 to 30.4 Bending actuator

What is claimed is:

1. A video endoscope comprising:
an endoscope shaft;
an optical assembly provided in a distal region of the endoscope shaft, the optical assembly being mounted in the endoscope shaft by a mounting; and
one or more reversibly deformable bending actuators operatively connected to the mounting such that the one or more bending actuators move the optical assembly relative to the endoscope shaft.

2. The video endoscope according to claim 1, wherein at least one of the one or more bending actuators are arranged between an inner wall of the endoscope shaft and the optical assembly.

3. The video endoscope according to claim 1, wherein the one or more bending actuators comprises two or more bending actuators arranged in the endoscope shaft one or more of symmetrically and uniformly relative to a longitudinal axis of the endoscope shaft.

4. The video endoscope according to claim 1, wherein the one or more bending actuators are connected at an end to the optical assembly to form an adhesive contact point between the one or more bending actuators and the optical assembly.

5. The video endoscope according to claim 1, wherein the mounting is one of an at least partially circular mounting body or an at least partially annular mounting body.

6. The video endoscope according to claim 1, wherein the one or more bending actuators are configured as one or more of planar, plate-shaped and strip-shaped.

7. The video endoscope according to claim 1, wherein the one or more bending actuators are configured as one of a piezo-electric bending actuator, a bending actuator with a shape memory alloy or with a bimetal arrangement.

8. The video endoscope according to claim 1, further comprising a controller configured to control the one or more bending actuators.

9. The video endoscope according to claim 1, wherein the one or more bending actuators comprise two or more bending actuators and the controller is configured to individually control each of the two or more bending actuators.

10. The video endoscope according to claim 1, wherein the optical assembly comprises at least one of a lens, a prism and an objective lens.

11. The video endoscope according to claim 1, wherein the optical assembly comprises an image sensor.

12. The video endoscope according to claim 11, wherein the image sensor is one of a CCD sensor or a CMOS sensor.

13. An optical assembly for use in an endoscope shaft of a video endoscope, the optical assembly comprising:
a mounting provided in a distal region of the endoscope shaft;
one or more optical elements mounted in the mounting; and
one or more reversibly deformable bending actuators operatively connected to the mounting such that the one or more bending actuators move the mounting relative to the endoscope shaft.
